SEQUENCE LISTING (5 sequences)

SEQ ID NO: 1 - DNA, 20 bp, Unknown
  Primer
taggagtttagtgaacttgc

SEQ ID NO: 2 - DNA, 26 bp, Unknown
  Primer
agcccttatccgatccactagaaaac

SEQ ID NO: 3 - DNA, 71 bp, Unknown
  Primer
gttttctagtggatcggataagggctccagaaccaactccagaaccaactctacctatccccgcacaaaac

SEQ ID NO: 4 - DNA, 18 bp, Unknown
  Primer
ttcgagcgtcccaaaacc

SEQ ID NO: 5 - DNA, 71 bp, Unknown
  Primer
gttttctagtggatcggataagggctgattacaaggatgacgatgacaagctacctatccccgcacaaaac

(19) United States
(12) Patent Application Publication
(10) Pub. No.: US 2006/0188888 A1
(43) Pub. Date: Aug. 24, 2006

(54) Method of screening for improved specific activity of enzymes

(75) Inventor: Shiro Fukuyama, Chiba (JP)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 10/564,179

(22) PCT Filed: Jul. 9, 2004
(86) PCT No.: PCT/DK04/00495

§ 371 Date: Jan. 6, 2006

(30) Foreign Application Priority Data
Jul. 11, 2003 (DK) PA 2003 01056

(51) Int. Cl. C12Q 1/68; C12Q 1/34; C12Q 1/37; C12Q 1/26; C40B 40/02; C40B 40/08

(52) U.S. Cl. 435/6; 435/18; 435/23; 435/25

(57) ABSTRACT

This invention relates to a method for screening libraries of enzyme variants for changes in specific activity by expression of a fusion protein consisting of at least two enzymes. By using one enzyme as a marker changes in specific activity for the other enzyme can be screened efficiently.

### FIELD OF INVENTION

This invention relates to a method for screening libraries of enzyme variants for changes in specific activity by expression of a fusion protein consisting of at least two enzymes. By using one enzyme as a marker changes in specific activity for the other enzyme can be screened efficiently.

### BACKGROUND OF THE INVENTION

Many methods of screening for improved characteristics of proteins, e.g. enzymes, have been reported. One property of enzymes which it is desirable to improve is the specific activity. Technologies such as DNA-shuffling and random or site-directed mutagenesis have allowed the production of large numbers of variants in a short time. It is therefore desirable to a method that allows for fast, eventually high through-put, screening of enzymes with modified specific activity.

### SUMMARY OF THE INVENTION

The problem to be solved by the present invention is to provide a method for perform screening for altered specific activity of an enzyme.

The problem arises because it is difficult to define the enzyme protein amount—and consequently to determine activity per milligram of enzyme protein—in the host cell culture supernatant without a purification process. To overcome this problem a method has been developed comprising the steps of
(i) generating a library of nucleic acid sequences encoding enzyme variants of interest
(ii) providing a nucleic acid sequence encoding an enzyme to be fused with the enzyme in (i)
(iii) fusing nucleic acid sequence encoding enzyme variants in (i) with nucleic acid sequence encoding enzyme in (ii)
(iv) transforming the fused nucleic acid sequence obtained in (iii) into a host cell
(v) culturing host cell in (iv) in order to express the fused enzymes
(vi) sampling each cell culture obtained in (v)
(vii) analyzing samples obtained in (vi) by determining activity ratio of the expressed fused enzymes
(viii) selecting the samples exhibiting the desired activity ratio.
   Definitions

Prior to a discussion of the detailed embodiments of the invention, a definition of specific terms related to the main aspects of the invention is provided.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”) DNA Cloning: A Practical Approach, Volumes I and II/D. N. Glover ed. 1985); Oligonucleotide Synthesis (M. J. Gait ed. 1984); Nucleic Acid Hybridization (B. D. Hames & S. J. Higgins eds (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984).

When applied to a protein, the term “isolated” indicates that the protein is found in a condition other than its native environment. In a preferred form, the isolated protein is substantially free of other proteins, i.e. more than 95% pure, more preferably more than 99% pure. When applied to a polynucleotide molecule, the term “isolated” indicates that the molecule is removed from its natural genetic milieu, and is thus free of other extraneous or unwanted coding sequences, and is in a form suitable for use within genetically engineered protein production systems. Such isolated molecules are those that are separated from their natural environment and include cDNA and genomic clones. Isolated DNA molecules of the present invention are free of other genes with which they are ordinarily associated, and may include naturally occurring 5′ and 3′ untranslated regions such as promoters and terminators. The identification of associated regions will be evident to one of ordinary skill in the art (see for example, Dynan and Tijan, Nature 316: 774-78, 1985).

A “polynucleotide” is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”) in either single stranded form, or a double-stranded helix. Double stranded DNA-DNA, DNA-RNA and RNA-RNA h slices are possible. The term nucleic add molecule, and in particular DNA or RNA molecule, refers only to the primary and secondary structure of the molecule, and does not limit it to any particular tertiary or quaternary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation.

A DNA “coding sequence” is a double-stranded DNA sequence, which is transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. If the coding sequence is intended for expression in a eukaryotic cell, a polyadenylation signal and transcription termination sequence will usually be located 3′ to the coding sequence.

A “gene” refers a nucleic acid sequence encoding a peptide, a polypeptide or a protein.

An “Expression vector” is a DNA molecule, linear or circular, that comprises a segment encoding a polypeptide of interest operably linked to additional segments that provide for its transcription. Such additional segments may include promoter and terminator sequences, and optionally one or more origins of replication, one or more selectable markers, an enhancer, a polyadenylation signal, and the like. Expression vectors are generally derived from plasmid or viral DNA, or may contain elements of both.

Transcriptional and translational control sequences are DNA regulatory sequences, such as promoters, enhancers, terminators, and the like, that provide for the expression of a coding sequence in a host cell. In eukaryotic cells, polyadenylation signals are control sequences.

A “secretory signal sequence” is a DNA sequence that encodes a polypeptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

“Operably linked”, when referring to DNA segments, indicates that the segments are arranged so that they function in concert for their intended purposes, e.g. transcription initiates in the promoter and proceeds through the coding segment to the terminator.

A coding sequence is “under the control” of transcriptional and translational control sequences in a cell when RNA polymerase transcribes the coding sequence into mRNA, which is then trans-RNA spliced and translated into the protein encoded by the coding sequence.

“Isolated polypeptide” is a polypeptide which is essentially free of other non-[enzyme] polypeptides, e.g., at least about 20% pure, preferably at least about 40% pure, more preferably about 60% pure, even more preferably about 80% pure, most preferably about 90% pure, and even most preferably about 95% pure, as determined by SDS-PAGE.

“Heterologous” DNA refers to DNA not naturally located in the cell, or in a chromosomal site of the cell. Preferably, the heterologous DNA includes a gene foreign to the cell.

A cell has been “transfected” by exogenous or heterologous DNA when such DNA has been introduced inside the cell. A cell has been “transformed” by exogenous or heterologous DNA when the transfected DNA effects a phenotypic change. Preferably, the transforming DNA should be integrated (covalently linked) into chromosomal DNA making up the genome of the cell.

“Homologous recombination” refers to the insertion of a foreign DNA sequence of a vector in a chromosome. Preferably, the vector targets a specific chromosomal site for homologous recombination. For specific homologous recombination, the vector will contain sufficiently long regions of homology to sequences of the chromosome to allow complementary binding and incorporation of the vector into the chromosome. Longer regions of homology, and greater degrees of sequence similarity, may increase the efficiency of homologous recombination.

“Specific activity” of an enzyme is activity unit per milligram of enzyme protein.

A “library” is a collection of entities having a common feature, e.g. a collection of nucleotide sequences encoding (different) enzymes.

The term “randomized library” of protein variants refers to a library with at least partially randomized composition of the members, e.g. protein variants.

The term “functionality” of protein variants refers to e.g. enzymatic activity, binding to a ligand or receptor, stimulation of a cellular response (e.g. 3H-thymidine incorporation as response to a mitogenic factor), or anti-microbial activity.

By the term “specific polyclonal antibodies” is meant polyclonal antibodies isolated according to their specificity for a certain antigen, e.g. the protein backbone.

“Spiked mutagenesis” is a form of site-directed mutagenesis, in which the primers used have been synthesized using mixtures of oligonucleotides at one or more positions.

### DETAILED DESCRIPTION OF THE INVENTION

The present invention relates to a method for screening enzyme variants for improved specific activity. The specific activity is altered by generating enzyme variants starting from a protein backbone, typically an enzyme. The improved specific activity of the enzyme variant may either be higher or lower than the specific activity found in the parent enzyme, i.e. protein backbone that has been modified, depending on the application of the enzyme variant.

Changes in specific activity of the generated enzyme variants are difficult to monitor without applying a purification step due to the presence of other proteins in the host cell culture supernatant. This problem has been solved in the present invention by constructing a fusion protein which consists of two enzymes. One of the enzymes in the fusion protein is the enzyme variant with changed specific activity, and the other enzyme is an enzyme with known specific activity (herein after the marker enzyme). Choice of marker enzyme depends on enzyme variant as the two enzymes preferably have no overlap in the analytical signal produced.

The present invention comprises the steps of:
(i) generating a library of nucleic acid sequences encoding enzyme variants of interest
(ii) providing a nucleic acid sequence encoding a marker enzyme to be fused with the enzyme in (i)
(iii) fusing nucleic acid sequence encoding enzyme variants in (i) with nucleic acid sequence encoding enzyme in (ii)
(iv) transforming the fused nucleic sequence obtained in (iii) into a host cell
(v) culturing host cell in (iv) in order to express the fused enzymes
(vi) sampling each cell culture obtained in (v)
(vii) analyzing samples obtained in (vi) by determining activity ratio of the expressed fused enzymes
(viii) selecting the samples exhibiting the desired activity ratio.
   Nucleic Acid Sequence

The techniques used to isolate or clone a nucleic acid sequence encoding a polypeptide are known in the art and include isolation from genomic DNA, preparation from cDNA, or a combination thereof. The cloning of the nucleic acid sequences of the present invention from such genomic DNA can be effected, e.g., by using the well known polymerase chain reaction (PCR) or antibody screening of expression libraries to detect cloned DNA fragments with shared structural features. See e.g. Innis et al., 1990, A Guide to Methods and Application, Academic Press, New York. Other nucleic acid amplification procedures such as ligase chain reaction (LCR), ligated activated transcription (LAT) and nucleic acid sequence-based amplification (NASBA) may be used. The nucleic acid sequence may be cloned from a strain producing the polypeptide, or from another related organism and thus, for example, may be an allelic or species variant of the polypeptide encoding region of the nucleic acid sequence.

The term “isolated” nucleic add sequence as used herein refers to a nucleic acid sequence which is essentially free of other nucleic acid sequences, e.g., at least about 20% pure, preferably at least about 40% pure, more preferably about 60% pure, even more preferably about 80% pure, most preferably about 90% pure, and even most preferably about 95% pure, as determined by agarose gel electorphoresis. For example, an isolated nucleic acid sequence can be obtained by standard cloning procedures used in genetic engineering to relocate the nucleic acid sequence from its natural location to a different site where it will be reproduced. The cloning procedures may involve excision and isolation of a desired nucleic acid fragment comprising the nucleic acid sequence encoding the polypeptide, insertion of the fragment into a vector molecule, and incorporation of the recombinant vector into a host cell where multiple copies or clones of the nucleic acid sequence will be replicated. The nucleic acid sequence may be of genomic, cDNA, RNA, semisynthetic, synthetic origin, or any combinations thereof.

Nucleic Add Construct

As used herein the term “nucleic add constructs is intended to indicate any nucleic acid molecule of cDNA, genomic DNA, synthetic DNA or RNA origin. The term “construct” is intended to indicate a nucleic acid segment which may be single- or double-stranded, and which may be based on a complete or partial naturally occurring nucleotide sequence encoding a polypeptide of interest. The construct may optionally contain other nucleic acid segments.

The DNA of interest may suitably be of genomic or cDNA origin, for instance obtained by preparing a genomic or cDNA library and screening for DNA sequences coding for all or part of the polypeptide by hybridization using synthetic oligonucleotide probes in accordance with standard techniques (cf. Sambrook et al., supra).

The nucleic acid construct may also be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. According to the phosphoamidite method, oligonucleotides are synthesized, e.g. In an automatic DNA synthesizer, purified, annealed, ligated and cloned in suitable vectors.

Furthermore, the nucleic acid construct may be of mixed synthetic and genomic, mixed synthetic and cDNA or mixed genomic and cDNA origin prepared by ligating fragments of synthetic, genomic or cDNA origin (as appropriate), the fragments corresponding to various parts of the entire nucleic acid construct, in accordance with standard techniques.

The nucleic acid construct may also be prepared by polymerase chain reaction using specific primers, for instance as described in U.S. Pat. No. 4,683,202 or Saiki et al., Science 239 (1988), 487-491.

The term nucleic acid construct may be synonymous with the term expression cassette when the nucleic acid construct contains all the control sequences required for expression of a coding sequence of the present invention. The term “coding sequence” as defined herein is a sequence which is transcribed into mRNA and translated into a polypeptide of the present invention when placed under the control of the above mentioned control sequences. The boundaries of the coding sequence are generally determined by a translation start codon ATG at the 5′-terminus and a translation stop codon at the 3′-terminus. A coding sequence can include, but is not limited to, DNA, cDNA, and recombinant nucleic acid sequences.

The term “control sequences” is defined herein to include all components which are necessary or advantageous for expression of the coding sequence of the nucleic acid sequence. Each control sequence may be native or foreign to the nucleic add sequence encoding the polypeptide. Such control sequences include, but are not limited to, a leader, a polyadenylation sequence, a propeptide sequence, a promoter, a signal sequence, and a transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleic add sequence encoding a polypeptide.

The control sequence may be an appropriate promoter sequence, a nucleic acid sequence which is recognized by a host cell for expression of the nucleic acid sequence. The promoter sequence contains transcription and translation control sequences which mediate the expression of the polypeptide. The promoter may be any nucleic acid sequence which shows transcriptional activity in the host cell of choice and may be obtained from genes encoding extracellular or intracellular polypeptides either homologous or heterologous to the host cell.

The control sequence may also be a suitable transcription terminator sequence, a sequence recognized by a host cell to terminate transcription. The terminator sequence is operably linked to the 3′ terminus of the nucleic acid sequence encoding the polypeptide. Any terminator which is functional in the host cell of choice may be used in the present invention.

The control sequence may also be a polyadenylation sequence, a sequence which is operably linked to the 3′ terminus of the nucleic acid sequence and which, when transcribed, is recognized by the host cell as a signal to add polyadenosine residues to transcribed mRNA. Any polyadenylation sequence which is functional in the host cell of choice may be used in the present invention.

The control sequence may also be a signal peptide coding region, which codes for an amino acid sequence linked to the amino terminus of the polypeptide which can direct the expressed polypeptide into the cell's secretory pathway of the host cell. The 5′ end of the coding sequence of the nucleic acid sequence may inherently contain a signal peptide coding region naturally linked in translation reading frame with the segment of the coding region which encodes the secreted polypeptide. Alternatively, the 5′ end of the coding sequence may contain a signal peptide coding region which is foreign to that portion of the coding sequence which encodes the secreted polypeptide. A foreign signal peptide coding region may be required where the coding sequence does not normally contain a signal peptide coding region. Alternatively, the foreign signal peptide coding region may simply replace the natural signal peptide coding region in order to obtain enhanced secretion relative to the natural signal peptide coding region normally associated with the coding sequence. The signal peptide coding region may be obtained from a glucoamylase or an amylase gene from an Aspergillus species, a lipase or proteinase gene from a Rhizomucor species, the gene for the alpha-factor from Saccharomyces cerevisiae, an amylase or a protease gene from a Bacillus species, or the calf preprochymosin gene. However, any signal peptide coding region capable of directing the expressed polypeptide into the secretory pathway of a host cell of choice may be used in the present invention.

The control sequence may also be a propeptide coding region, which codes for an amino acid sequence positioned at the amino terminus of a polypeptide. The resultant polypeptide is known as a proenzyme or propolypeptide (or a zymogen in some cases). A propolypeptide is generally inactive and can be converted to mature active polypeptide by catalytic or autocatalytic cleavage of the propeptide from the propolypeptide. The propeptide coding region may be obtained from the Bacillus subtilis alkaline protease gene (aprE), the Bacillus subtilis neutral protease gene (nprT), the Saccharomyces cerevisiae alpha-factor gene, or the Myceliophthora thermophilum laccase gene (WO 95/33836).

The nucleic acid constructs of the present invention may also comprise one or more nucleic acid sequences which encode one or more factors that are advantageous in the expression of the polypeptide, e.g., an activator (e.g., a trans-acting factor), a chaperone, and a processing protease. Any factor that is functional in the host cell of choice may be used in the present invention. The nucleic acids encoding one or more of these factors are not necessarily in tandem with the nucleic acid sequence encoding the polypeptide.

An activator is a protein which activates transcription of a nucleic acid sequence encoding a polypeptide (Kudla et al., 1990, EMBO Journal 9:1355-1364; Jaral and Buxton, 1994, Current Genetics 26:2238-244; Verdler, 1990, Yeast 6:271-297). The nucleic acid sequence encoding an activator may be obtained from the genes encoding Bacillus stearothermophilus NprA (nprA), Saccharomyces cerevisiae heme activator protein 1 (hap1), Saccharomyces cerevisiae galactose metabolizing protein 4 (gal4), and Aspergillus nidulans ammonia regulation protein (areA). For further examples, see Verdier, 1990, supra and MacKenzie et al., 1993, Journal of General Microbiology 139:2295-2307.

A chaperone is a protein which assists another polypeptide in folding properly (Hartl et al., 1994, TIBS 19:20-25; Bergeron et al., 1994, TIBS 19:124-128; Demolder et al., 1994, Journal of Biotechnology 32:179-189; Craig, 1993, Science 260:1902-1903; Gething and Sambrook, 1992, Nature 355:3345; Puig and Gilbert, 1994, Journal of Biological Chemistry 269:7764-7771; Wang and Tsou, 1993, The FASEB Journal 7:1515-11157; Robinson et al., 1994, Bio/Technology 1:381-384). The nucleic acid sequence encoding a chaperone may be obtained from the genes encoding Bacillus subtilis GroE proteins, Aspergillus oryzae protein disulphide isomerase, Saccharomyces cerevisiae calnexin, Saccharomyces cerevisiae BIP/GRP78, and Saccharomyces cerevisiae Hsp70. For further examples, see Gething and Sambrook, 1992, supra, and Harti et al., 1994, supra.

A processing protease is a protease that cleaves a propeptide to generate a mature biochemically active polypeptide (Enderin and Ogrydzlak, 1994, Yeast 10:67-79; Fuller et al., 1989, Proceedings of the National Academy of Sciences USA 86:1434-1438; Julius et al., 1984, Cell 37:1075-1089; Julius et al., 1983, Cell 32:839-852). The nucleic acid sequence encoding a processing protease may be obtained from the genes encoding Aspergillus niger Kex2, Saccharomyces cerevisiae dipeptidylaminopeptidase, Saccharomyces cerevsiae Kex2, and Yarrowia lipolytica dibasic processing endoprotease (xpr6).

It may also be desirable to add regulatory sequences which allow the regulation of the expression of the polypeptide relative to the growth of the host cell. Examples of regulatory systems are those which cause the expression of the gene to be turned on or off in response to a chemical or physical stimulus, including the presence of a regulatory compound. Regulatory systems in prokaryotic systems would include the lac, tac, and trp operator systems. In yeast, the ADH2 system or GALL system may be used. In filamentous fungi, the TAKA alpha-amylase promoter, Aspergillus niger glucoamylase promoter, and the Aspergillus oryzae glucoamylase promoter may be used as regulatory sequences. Other examples of regulatory sequences are those which a low for gene amplification. In eukaryotic systems, these include the dihydrofolate reductase gene which is amplified in the presence of methotrexate, and the metallothionein genes which are amplified with heavy metals. In these cases, the nucleic acid sequence encoding the polypeptide would be placed in tandem with the regulatory sequence.

Nucleic Acid Sequence Library

Preparation of a nucleic acid sequence library can be achieved by use of known methods.

Procedures for extracting genes from a cellular nucleotide source and preparing a gene library are described in e.g. Pitcher et al., “Rapid extraction of bacterial genomic DNA with guanidium thiocyanate”, Lett. Appl. Microbiol., 8, pp 151-156, 1989, Dretzen, G. et al., “A reliable method for the recovery of DNA fragments from agarose and acrylamide gels”, Anal. Biochem., 112, pp 295-298, 1981, WO 94/19454 and Diderichsen et al., “Cloning of aldB, which encodes alpha-acetolactate decarboxylase, an exoenzyme from Bacillus brevis”, J. Bacteriol., 172, pp 43154321,1990.

Procedures for preparing a gene library from an in vitro made synthetic nucleotide source can be found in (e.g. described by Stemmer, Proc. Natl. Acad. Sci. USA, 91, pp. 10747-10751, 1994 or WO 95/17413).

Promoters

Examples of suitable promoters for directing the transcription of the nucleic acid constructs of the present invention, especially in a bacterial host cell, are the promoters obtained from the E. coli lac operon, the Streptomyces coelicolor agarase gene (dagA), the Bacillus subtilis levansucrase gene (sacB), the Bacillus subtilis alkaline protease gene, the Bacillus licheniformis alpha-amylase gene (amyL), the Bacillus stearothermophilus maltogenic amylase gene (amyM), the Bacillus amyloliquefaciens alpha-amylase gene (amyQ), the Bacillus amyloliquefaciens BAN amylase gene, the Bacillus licheniformis penicillinase gene (penP), the Bacillus subtilis xyIA and xyIB genes, and the prokaryotic beta-lactamase gene (Villa-Kamaroff et al., 1978, Proceedings of the National Academy of Sciences USA 75:3727-3731), as well as the tac promoter (DeBoer et al., 1983, Proceedings of the National Academy of Sciences USA 80:21-25), or the Bacillus pumilus xylosidase gene, or by the phage Lambda PR or PL promoters or the E. coli lac, trp or tac promoters. Further promoters are described in “Useful proteins from recombinant bacteria” in Scientific American, 1980, 242:74-94; and in Sambrook et al., 1989, supra.

Examples of suitable promoters for directing the transcription of the nucleic acid constructs of the present invention in a filamentous fungal host cell are promoters obtained from the genes encoding Aspergillus oryzae TAKA amylase, Rhizomucor miehei aspartic proteinase, Aspergillus niger neutral alpha amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, Fusarium oxysporum trypsin-like protease (as described in U.S. Pat. No. 4,288,627, which is incorporated herein by reference), and hybrids thereof. Particularly preferred promoters for use in filamentous fungal host cells are the TAKA amylase, NA2-tpi (a hybrid of the promoters from the genes encoding Aspergillus niger neutral (amylase and Aspergillus oryzae triose phosphate isomerase), and glaA promoters. Further suitable promoters for use in filamentous fungus host cells are the ADH3 promoter (McKnight et al., The EMBO J. 4 (1985), 2093-2099) or the tpiA promoter.

Examples of suitable promoters for use in yeast host cells include promoters from yeast glycolytic genes (Hitzeman et al., J. Biol. Chem. 255 (1980), 12073-12080; Alber and Kawasaki, J. Mol. Appl. Gen. 1 (1982), 419-434) or alcohol dehydrogenase genes (Young et al., in Genetic Engineering of Microorganisms for Chemicals (Hollaender et al, eds.), Plenum Press, New York, 1982), or the TPI1 (U.S. Pat. No. 4,599,311) or ADH2-4c (Russell et al., Nature 304 (1983), 652-654) promoters.

Further useful promoters are obtained from the Saccharomyces cerevisiae enolase (ENO-1) gene, the Saccharomyces cerevisiae galactokinase gene (GALI), the Saccharomyces cerevisiae alcohol dehydrogenase/glyceraldehyde-3-phosphate dehydrogenase genes (ADH2/GAP), and the Saccharomyces cerevisiae 3-phosphoglycerate kinase gene. Other useful promoters for yeast host cells are described by Romanos et al., 1992, Yeast 8:423-488. In a mammalian host cell, useful promoters include viral promoters such as those from Simian Virus 40 (SV40), Rous sarcoma virus (RSV), adenovirus, and bovine papilloma virus (BPV).

Examples of suitable promoters for directing the transcription of the DNA encoding the polypeptide of the invention in mammalian cells are the SV40 promoter (Subramani et al., Mol. Cell Biol. 1 (1981), 854-864), the MT-1 (metallothionein gene) promoter (Palmiter et al., Science 222 (1983), 809-814) or the adenovirus 2 major late promoter.

An example of a suitable promoter for use in insect cells is the polyhedrin promoter (U.S. Pat. No. 4,745,051; Vasuvedan et al., FEBS Lett. 311, (1992) 7-11), the P10 promoter (J. M. Vlak et al., J. Gen. Virology 69, 1988, pp. 765-776), the Autographa californica polyhedrosis virus basic protein promoter (EP 397 485), the baculovirus immediate early gene 1 promoter (U.S. Pat. No. 5,155,037; U.S. Pat. No. 5,162,222), or the baculovirus 39K delayed-early gene promoter (U.S. Pat. No. 5,155,037; U.S. Pat. No. 5,162,222).

Terminators

Preferred terminators for filamentous fungal host cells are obtained from the genes encoding Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. for fungal hosts) the TPI1 (Alber and Kawasaki, op. cit.) or ADH3 (McKnight et al., op. cit.) terminators.

Preferred terminators for yeast host cells are obtained from the genes encoding Saccharomyces cerevisiae enolase, Saccharomyces cerevisiae cytochrome C (CYCI), or Saccharomyces cerevisiae glyceraldehyde-3-phosphate dehydrogenase. Other useful terminators for yeast host cells are described by Romanos et al., 1992, supra.

Polyadenylation Signals

Preferred polyadenylation sequences for filamentous fungal host cells are obtained from the genes encoding Aspergillus oryzae TAKA amylase, Aspergillus niger glucoamylase, Aspergillus nidulans anthranilate synthase, and Aspergillus niger alpha-glucosidase. Useful polyadenylation sequences for yeast host cells are described by Guo and Sherman, 1995, Molecular Cellular Biology 15:5983-5990.

Signal Sequences

An effective signal peptide coding region for bacterial host cells is the signal peptide coding region obtained from the maltogenic amylase gene from Bacillus NCIB 11837, the Bacillus stearothermophilus alpha-amylase gene, the Bacillus licheniformis subtilisin gene, the Bacillus licheniformis beta-lactamase gene, the Bacillus stearothermophilus neutral proteases genes (nprT, nprS, nprM), and the Bacillus subtilis PrsA gene. Further signal peptides are described by Simonen and Palva, 1993, Microbiological Reviews 57:109-137.

An effective signal peptide coding region for filamentous fungal host cells is the signal peptide coding region obtained from Aspergillus oryzae TAKA amylase gene, Aspergillus niger neutral amylase gene, the Rhizomucor miehei aspartic proteinase gene, the Humicola lanuginosa cellulase or lipase gene, or the Rhizomucor miehei lipase or protease gene, Aspergillus sp. amylase or glucoamylase, a gene encoding a Rhizomucor miehei lipase or protease. The signal peptide is preferably derived from a gene encoding A. oryzae TAKA amylase, A. niger neutral alfa-amylase, A. niger acid-stable amylase, or A. niger glucoamylase.

Useful signal peptides for yeast host cells are obtained from the genes for Saccharomyces cerevisiae a-factor and Saccharomyces cerevisiae invertase. Other useful signal peptide coding regions are described by Romanos et al., 1992, supra.

For secretion from yeast cells, the secretory signal sequence may encode any signal peptide which ensures efficient direction of the expressed polypeptide into the secretory pathway of the cell. The signal peptide may be a naturally occurring signal peptide, or a functional part thereof, or it may be a synthetic peptide. Suitable signal peptides have been found to be the a-factor signal peptide (cf. U.S. Pat. No. 4,870,008), the signal peptide of mouse salivary amylase (cf. O. Hagenbuchle et al., Nature 289, 1981, pp. 643-646), a modified carboxypeptidase signal peptide (cf. L. A. Valls et al., Cell 48, 1987, pp. 887-897), the yeast BAR1 signal peptide (cf. WO 87/02670), or the yeast aspartic protease 3 (YAP3) signal peptide (cf. M. Egel-Mitani et al., Yeast 6, 1990, pp. 127-137).

For efficient secretion in yeast, a sequence encoding a leader peptide may also be inserted downstream of the signal sequence and uptream of the DNA sequence encoding the polypeptide. The function of the leader peptide is to allow the expressed polypeptide to be directed from the endoplasmic reticulum to the Golgl apparatus and further to a secretory vesicle for secretion into the culture medium (i.e. exportation of the polypeptide across the cell wall or at least through the cellular membrane into the periplasmic space of the yeast cell). The leader peptide may be the yeast a-factor leader (the use of which is described in e.g. U.S. Pat. No. 4,546,082, EP 16 201, EP 123 294, EP 123 544 and EP 163 529). Alternatively, the leader peptide may be a synthetic leader peptide, which is to say a leader peptide not found in nature. Synthetic leader peptides may, for instance, be constructed as described in WO 89/02463 or WO 92/11378.

Expression Vectors

The present invention also relates to recombinant expression vectors comprising a nucleic acid sequence of the present invention, a promoter, and transcriptional and translational stop signals. The various nucleic acid and control sequences described above may be joined together to produce a recombinant expression vector which may include one or more convenient restriction sites to allow for insertion or substitution of the nucleic acid sequence encoding the polypeptide at such sites. Alternatively, the nucleic acid sequence of the present invention may be expressed by inserting the nucleic acid sequence or a nucleic acid construct comprising the sequence into an appropriate vector for expression. In creating the expression vector, the coding sequence is located in the vector so that the coding sequence is operably linked with the appropriate control sequences for expression, and possibly secretion.

The recombinant expression vector may be any vector (e.g., a plasmid or virus) which can be conveniently subjected to recombinant DNA procedures and can bring about the expression of the nucleic acid sequence. The choice of the vector will typically depend on the compatibility of the vector with the host cell into which the vector is to be introduced. The vectors may be linear or closed circular plasmids. The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. The vector system may be a single vector or plasmid or two or more vectors or plasmids which together contain the total DNA to be introduced into the genome of the host cell, or a transposon.

The vectors of the present invention preferably contain one or more selectable markers which permit easy selection of transformed cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers which confer antibiotic resistance such as ampicillin, kanamycin, chloramphenicol, tetracycline, neomycin, hygromycin or methotrexate resistance.

Suitable markers for yeast host cells are ADE2, HIS3, LEU2, LYS2, MET3, TRP1, and URA3. A selectable marker for use in a filamentous fungal host cell may be selected from the group including, but not limited to, amdS (acetamidase), argB (ornithine arbamoyltransferase), bar (phosphinothricin acetyltransferase), hygB (hygromycin phosphotransferase), niaD (nitrate reductase), pyrG (orotidine-5′-phosphate decarboxylase), sC (sulfate adenyltransferase), trpC (anthranilate synthase), and glufosinate resistance markers, as well as equivalents from other species. Preferred for use in an Aspergillus cell are the amdS and pyrG markers of Aspergillus nidulans or Aspergillus oryzae and the bar marker of Streptomyces hygroscopicus. Furthermore, selection may be accomplished by co-transformation, e.g., as described in WO 91/17243, where the selectable marker is on a separate vector.

The vectors of the present invention preferably contain an element(s) that permits stable integration of the vector into the host cell genome or autonomous replication of the vector in the cell independent of the genome of the cell.

The vectors of the present invention may be integrated into the host cell genome when introduced into a host cell. For integration, the vector may rely on the nucleic acid sequence encoding the polypeptide or any other element of the vector for stable integration of the vector into the genome by homologous or nonhomologous recombination. Alternatively, the vector may contain additional nucleic acid sequences for directing integration by homologous recombination into the genome of the host cell. The additional nucleic acid sequences enable the vector to be integrated into the host cell genome at a precise location(s) in the chromosome(s). To increase the likelihood of integration at a precise location, the integrational elements should preferably contain a sufficient number of nucleic acids, such as 100 to 1,500 base pairs, preferably 400 to 1,500 base pairs, and most preferably 800 to 1,500 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleic acid sequences. On the other hand, the vector may be integrated into the genome of the host cell by non-homologous recombination. These nucleic acid sequences may be any sequence that is homologous with a target sequence in the genome of the host cell, and, furthermore, may be non-encoding or encoding sequences.

For autonomous replication, the vector may further comprise an origin of replication enabling the vector to replicate autonomously in the host cell in question. Examples of bacterial origins of replication are the origins of replication of plasmids pBR322, pUC19, pACYC177, pACYC184, pUB110, pE194, pTA1060, and pAMB1. Examples of origin of replications for use in a yeast host cell are the 2 micron origin of replication, the combination of CEN6 and ARS4, and the combination of CEN3 and ARS1. The origin of replication may be one having a mutation which makes its functioning temperature-sensitive in the host cell (see, e.g., Ehrlich, 1978, Proceedings of the National Academy of Sciences USA 75:1433).

More than one copy of a nucleic acid sequence encoding a polypeptide of the present invention may be inserted into the host cell to amplify expression of the nucleic acid sequence. Stable amplification of the nucleic acid sequence can be obtained by integrating at least one additional copy of the sequence into the host cell genome using methods well known in the art and selecting for transformants.

The procedures used to ligate the elements described above to construct the recombinant expression vectors of the present invention are well known to one skilled in the art (see, e.g., Sambrook et al., 1989, supra).

Host Cells

The present invention also relates to recombinant host cells, comprising a nucleic acid sequence of the invention, which are advantageously used in the recombinant production of the polypeptides. The term “host cell” encompasses any progeny of a parent cell which is not identical to the parent cell due to mutations that occur during replication.

The cell is preferably transformed with a vector comprising a nucleic acid sequence of the invention followed by integration of the vector into the host chromosome. “Transformation” means introducing a vector comprising a nucleic acid sequence of the present invention into a host cell so that the vector is maintained as a chromosomal integrant or as a self-replicating extra-chromosomal vector. Integration is generally considered to be an advantage as the nucleic acid sequence is more likely to be stably maintained in the cell. Integration of the vector into the host chromosome may occur by homologous or non-homologous recombination as described above.

The choice of a host cell will to a large extent depend upon the gene encoding the polypeptide and its source. The host cell may be a unicellular microorganism, e.g., a prokaryote, or a non-unicellular microorganism, e.g., a eukaryote. Useful unicellular cells are bacterial cells such as gram positive bacteria including, but not limited to, a Bacillus cell, e.g., Bacillus alkalophilus, Bacillus amyloliquefaciens, Bacillus brevis, Bacillus circulans, Bacillus coagulans, Bacillus lautus, Bacillus lentus, Bacillus llcheniformis, Bacillus megaterium, Bacillus stearothermophilus, Bacillus subtilis, and Bacillus thuringiensis; or a Streptomyces cell, e.g., Streptomyces lividans or Streptomyces murinus, or gram negative bacteria such as E. coli and Pseudomonas sp. In a preferred embodiment, the bacterial host cell is a Bacillus lentus, Bacillus licheniformis, Bacillus stearothermophilus or Bacillus subtilis cell. The transformation of a bacterial host cell may, for instance, be effected by protoplast transformation (see, e.g., Chang and Cohen, 1979, Molecular General Genetics 168:111-115), by using competent cells (see, e.g., Young and Spizizin, 1961, Journal of Bacteriology 81:823-829, or Dubnar and Davidoff-Abelson, 1971, Journal of Molecular Biology 56:209-221), by electroporation (see, e.g., Shigekawa and Dower, 1988, Biotechniques 6:742-751), or by conjugation (see, e.g., Koehler and Thorne, 1987, Journal of Bacteriology 169:5771-5278).

The host cell may be a eukaryote. In a preferred embodiment, the host cell is a fungal cell. “Fungi” as used herein includes the phyla Ascomycota, Basidiomycota, Chytridlomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra). Representative groups of Ascomycota include, e.g., Neurospora, Eupenicillium (=Penicillium), Emericella (=Aspergillus), Eurotium (=Aspergillus), and the true yeasts listed above. Examples of Basidiomycota include mushrooms, rusts, and smuts. Representative groups of Chytridiomycota include, e.g., Allomyces, Blastocladiella, Coelomomyces, and aquatic fungi. Representative groups of Oomycota include, e.g., Saprolegniomycetous aquatic fungi (water molds) such as Achlya. Examples of mitosporic fungi include Aspergillus, Penicillium, Candida, and Altemaria. Representative groups of Zygomycota include, e.g., Rhizopus and Mucor.

In a preferred embodiment, the fungal host cell is a yeast cell. “Yeast” as used herein includes ascosporogenous yeast (Endomycetales), basidiosporogenous yeast, and yeast belonging to the Fungi Imperfecti (Blastomycetes). The ascosporogenous yeasts are divided into the families S permophthoraceae and S accharomycetaceae. The latter is comprised of four subfamilies, Schizosaccharomycoideae (e.g., genus Schizosaccharomyces), Nadsonioideae, Lipomycoideae, and Saccharomycoldeae (e.g., genera Pichia, Kluyveromyces and Saccharomyces). The basidiosporogenous yeasts include the genera Leucosporidim, Rhodosporidium, Sporidiobolus, Filobasidium, and Filobasidielia. Yeast belonging to the Fungi Imperfecti are divided into two families, Sporobolomycetaceae (e.g., genera Sorobolomyces and Bullera) and Cryptoooocaceae (e.g., genus Candida). Since the classification of yeast may change in the future, for the purposes of this invention, yeast shall be defined as described in biology and Activities of Yeast (Skinner, F. A., Passmore, S. M., and Davenport, R. R., eds, Soc. App. Bacteriol. Symposium Series No. 9, 1980. The biology of yeast and manipulation of yeast genetics are well known in the art (see, e.g., Biochemistry and Genetics of Yeast, Bacil, M., Horecker, B. J., and Stopani, A. O. M., editors, 2nd edition, 1987; The Yeasts, Rose, A. H., and Harrison, J. S., editors, 2nd edition, 1987; and The Molecular Biology of the Yeast Saccharomyces, Strathern et al., editors, 1981).

The yeast host cell may be selected from a cell of a species of Candida, Kluyveromyces, Saccharomyces, Schizosaocharomyces, Candida, Pichia, Hansehula, or Yarrowia. In a preferred embodiment, the yeast host cell is a Saccharomyces carlsbergensis, Saccharomyces cerevisiae, Saccharomyces diastaticus, Saccharomyces douglasii, Saccharomyces kluyveri, Saccharomyces norbensis or Saccharomyces oviformis cell. Other useful yeast host cells are a Kluyveromyces lactis Kluyveromyces fragilis Hansehula polymorpha, Pichia pastoris Yarrowia lipolytica, Schizosaccharomyces pombe, Ustilgo maylis, Candida maltose, Pichia guillermondii and Pichia methanolio cell (cf. Gleeson et al., J. Gen. Microbiol. 132, 1986, pp. 3459-3465; U.S. Pat. No. 4,882,279 and U.S. Pat. No. 4,879,231).

In a preferred embodiment, the fungal host cell is a filamentous fungal cell. Filamentous fungi” include all filamentous forms of the subdivision Eumycota and Oomycota (as defined by Hawksworth et al., 1995, supra). The filamentous fungi are characterized by a vegetative mycelium composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative. In a more preferred embodiment, the filamentous fungal host cell is a cell of a species of, but not limited to, Acremonium, Aspergillus, Fusarium, Humicola, Mucor, Myceliophthora, Neurospora, Penicillium, Thielavia, Tolypocladium, and Trichoderma or a teleomorph or synonym thereof. In an even more preferred embodiment, the filamentous fungal host cell is an Aspergillus cell. In another even more preferred embodiment, the filamentous fungal host cell is an Acremonium cell. In another even more preferred embodiment, the filamentous fungal host cell is a Fusarium cell. In another even more preferred embodiment, the filamentous fungal host cell is a Humicola cell. In another even more preferred embodiment, the filamentous fungal host cell is a Mucor cell. In another even more preferred embodiment, the filamentous fungal host cell is a Myceliophthora cell. In another even more preferred embodiment, the filamentous fungal host cell is a Neurospora cell. In another even more preferred embodiment, the filamentous fungal host cell is a Penicillium cell. In another even more preferred embodiment, the filamentous fungal host cell is a Thielavia cell. In another even more preferred embodiment, the filamentous fungal host cell is a Tolypociadium cell. In another even more preferred embodiment, the filamentous fungal host cell is a Trichoderma cell. In a most preferred embodiment, the filamentous fungal host cell is an Aspergillus awamori, Aspergillus foetidus, Aspergillus japonicus, Aspergillus niger, Aspergillus nidulans or Aspergillus oryzae cell. In another most preferred embodiment, the filamentous fungal host cell is a Fusarium cell of the section Discolor (also known as the section Fusarium). For example, the filamentous fungal parent cell may be a Fusarium bactridioides, Fusarium cerealls, Fusarium crookwellense, Fusarium culmorum, Fusarium graminearum, Fusarium graminum, Fusarium heterosporum, Fusarium negundi, Fusarium reticulatum, Fusarium roseum, Fusarium sambucinum, Fusarium sarcochroum, Fusarium sulphureum, or Fusarium trichothecloides cell. In another prefered embodiment, the filamentous fungal parent cell is a Fusarium strain of the section Elegans, e.g., Fusarium oxysporum. In another most preferred embodiment, the filamentous fungal host cell is a Humicola insolens or Humicola lanuginosa cell. In another most preferred embodiment, the filamentous fungal host cell is a Mucor miehei cell. In another most preferred embodiment, the filamentous fungal host cell is a Mycellophthora thermophilum cell. In another most preferred embodiment, the filamentous fungal host cell is a Neurospora crassa cell. In another most preferred embodiment, the filamentous fungal host cell is a Penicillium purpurogenum cell. In another most preferred embodiment, the filamentous fungal host cell is a Thielavia terrestris cell or a Acremonium chrysogenum cell. In another most preferred embodiment, the Trichoderma cell is a Trichoderma harzianum, Trichoderma koningii, Trichoderma longibrachiatum, Trichoderma reesei or Trichoderma viride cell. The use of Aspergillus spp. for the expression of proteins is described in, e.g., EP 272 277, EP 230 023.

Transformation

Fungal cells may be transformed by a process involving protoplast formation, transformation of the protoplasts, and regeneration of the cell wall in a manner known per se. Suitable procedures for transformation of Aspergillus host cells are described in EP 238 023 and Yelton et al., 1984, Proceedings of the National Academy of Sciences USA 81:1470-1474. A suitable method of transforming Fusarium species is described by Malardier et al., 1989, Gene 78:147-156 or in copending U.S. Ser. No. 08/269,449. Examples of other fungal cells are cells of filamentous fungi, e.g. Aspergillus spp., Neurospora spp., Fusarium spp. or Trichoderma spp., in particular strains of A. oryzae, A. nidulans or A. niger. The use of Aspergillus spp. for the expression of proteins is described in, e.g., EP 272 277, and EP 230 023. The transformation of F. oxysporum may, for instance, be carried out as described by Malardier et al., 1989, Gene 78: 147-156.

Yeast may be transformed using the procedures described by Becker and Guarente, in Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153:163; and Hinnen et al., 1978, Proceedings of the National Academy of Sciences USA 75:1920. Mammalian cells may be transformed by direct uptake using the calcium phosphate precipitation method of Graham and Van der Eb (1978, Virology 52:546).

Manipulating the Nucleic Acid Sequences of a Library

In a particular embodiment the genes of a gene library may before, during or after initiating the screening be subjected to alterations and or mutations by genetic engineering. Generation of libraries of genes encoding variants of enzymes can be done in a variety of ways:

(1) Error prone PCR employs a low fidelity replication step to introduce random point mutations at each round of amplification (Caldwell and Joyce (1992), PCR Methods and Applications vol. 2 (1), pp. 28-33). Error-prone PCR mutagenesis is performed using a plasmid encoding the wild-type, i.e. wt, gene of interest as template to amplify this gene with flanking primers under PCR conditions where increased error rates leads to introduction of random point mutations. The PCR conditions utilized are typically: 10 mM Tris-HCl, pH 8.3, 50 mM KCl, 4 mM MgCl2, 0.3 mM MnCl2, 0.1 mM dGTP/dATP, 0.5 mM dTTP/dCTP, and 2.5 u Taq polymerase per 100 micro L of reaction. The resultant PCR fragment is purified on a gel and cloned using standard molecular biology techniques.

(2) Oligonucleotide directed mutagenesis in single codon position (including deletions or insertions), e.g. by SOE-PCR is described by Kirchhoff and Desrosiers, PCR Methods and Applications, 1993, 2, 301-304. This method is performed as follows: Two independent PCR reactions are performed with 2 internal, overlapping primers, wherein one or both contain a mutant sequence and 2 external primers, which may encode restriction sites, thereby creating 2 overlapping PCR fragments. These PCR fragments are purified, diluted, and mixed in molar ratio 1:1. The full length PCR product is subsequently obtained by PCR amplification with the external primers. The PCR fragment is purified on gel and cloned using standard molecular biology techniques.

(3) Oligonucleotide directed randomization in single codon position, such as saturation mutagenesis, may be done e.g. by SOE-PCR as described above, but using primers with randomized nucleotides. For example NN(G/T), wherein N is any of the 4 bases G, A, T or C, will yield a mixture of codons encoding all possible amino acids.

(4) Combinatorial site-directed mutagenesis libraries may be employed, where several codons can be mutated at once using (2) and (3) above. For multiple sites, several overlapping PCR fragments are assembled simultaneously in a SOE-PCR setup.

(5) Another protocol employs synthetic gene libraries preparation. Wild type, i.e. wt, genes can be assembled from multiple overlapping oligonucleotides (typically 40-100 nucleotides in length; (Stemmer et al., (1995), Gene 164,49-53). By including mixtures of wt and mutant variants of the same oligo at various positions in the gene, the resulting assembled gene will contain mutations at various positions with mutagenic rates corresponding to the ratios of wt to mutant primers.

(6) Still another method employs multiple mutagenic primers to generate libraries with multiple mutated positions. First an uracil-containing nucleotide template encoding a polypeptide of interest is generated and 2-50 mutagenic primers corresponding to at least one region of identity in the nucleotide template are synthezised so that each mutagenic primer comprises at least one substitution of the template sequence (or insertion/deletion of bases) resulting in at least one amino acid substitution (or insertion/deletion) of the amino acid sequence encoded by the uracil-containing nucleotide template. The mutagenic primers are then contacted with the uracil-containing nucleotide template under conditions wherein a mutagenic primer anneals to the template sequence. This is followed by extension of the primer(s) catalyzed by a polymerase to generate a mixture of mutagenized polynucleotides and uracil-containing templates. Finally, a host cell is transformed with the polynucleotide and template mixture wherein the template is degraded and the mutagenized polynucleotide replicated, generating a library of polynucleotide variants of the gene of interest.

(7) Libraries may be created by shuffling e.g. by recombination of two or more wt genes or genes encoding variant proteins created by any combination of methods (1)-(6) (above) by DNA shuffling.

Fusion Protein

Fusion protein consists of two proteins which are connected, possibly by a linker peptide. The fusion protein has two functions originated from each protein (e.g. enzyme activity, anti-microbial activity). The two various nucleic acid sequence of two proteins may be joined together with a linker nucleic acid sequence by PCR technique, ligation or in vivo recombination.

Polypeptide Linker

The fusion protein of the present invention preferably contains one polypeptide linker which gives the proper flexibility to permit both proteins' activity expression. The linker sequence may be any linker which can connect two proteins covalently. The length of the linker depends on the target protein itself (e.g. stability, hydrophobicity). Examples of linkers, but not limited to these linkers, are Poly-Arg, Poly-His, PEPTPEPT, FLAG, Strep-tag II, c-myc, S—, HAT-, 3× FLAG, Calmoludin-binding peptide, Cellulose-binding domain, SBP, Chitin-binding domain, Glutathione S-transferase, Maltose-binding domain (see Terpe, K., 2003, Applied Microbiology and Biotechnology, 60(5):523-533).

Methods of Production

The transformed or transfected host cells described above are cultured in a suitable nutrient medium under conditions permitting the production of the desired molecules, after which these are recovered from the cells, or the culture broth.

The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The media are prepared using procedures known in the art (see, e.g., references for bacteria and yeast; Bennett, J. W. and LaSure, L., editors, More Gene Manipulations In Fungi, Academic Press, CA, 1991).

The cells may be cultured in any suitable container-unit, e.g. a shake flask, 24 well plates, 96 well plates, 384 well plates, 1536 well plates, or a higher number of wells per plate, or nanoliter well-less compartments.

In order to increase the number of individual activity assays performed in a given time the activity may conveniently be assayed in a high-throughput screening system using 96 well plates, 384 well plates, 1536 well plates, or a higher number of wells per plate, or nanoliter well-less compartments. Such screening techniques are well known in the art, see e.g. Dove, A., Nature Biotechnology (17), 1999, 859-863, and Kell, D., trends in Biotechnology (17), 1999, 89-91.

If the molecules are secreted into the nutrient medium, they can be recovered directly from the medium. If they are not secreted, they can be recovered from cell lysates. The molecules are recovered from the culture medium by conventional procedures including separating the host cells from the medium by centrifugation or filtration, precipitating the proteinaceous components of the supernatant or filtrate by means of a salt, e.g. ammonium sulphate, purification by a variety of chromatographic procedures, e.g. ion exchange chromatography, gelfiltration chromatography, affinity chromatography, or the like, dependent on the type of molecule in question.

The molecules of interest may be detected using methods known in the art that are specific for the molecules. These detection methods may include use of specific antibodies, formation of a product, or disappearance of a substrate. For example, an enzyme assay may be used to determine the activity of the molecule. Procedures for determining various kinds of activity are known in the art.

The molecules of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing (IEF), differential solubility (e.g., ammonium sulfate precipitation), or extraction (see, e.g., Protein Purification, J-C Janson and Lars Ryden, editors, VCH Publishers, New York, 1989).

The terms “relevant protein backbone” or “protein backbone” refer to the polypeptide to be modified by creating a library of diversified mutants. The “relevant protein backbone” may be a naturally occurring (or wild-type) polypeptide or it may be a variant thereof prepared by any suitable means. For instance, the “relevant protein backbone” may be a variant of a naturally occurring polypeptide which has been modified by substitution, deletion or truncation of one or more amino acid residues or by addition or insertion of one or more amino acid residues to the amino acid sequence of a naturally-occurring polypeptide.

In the present invention the enzyme to be varied as well as the marker enzyme may be selected from the group of enzymes comprising glycosyl hydrolases, carbohydrases, peroxidases, proteases, lipases, phytases, polysaccharide lyases, oxidoreductases, transglutaminases and glycoseisomerases, in particular the following.

Parent Proteases

Parent proteases (i.e. enzymes classified under the Enzyme Classification number E.C. 3.4 in accordance with the Recommendations (1992) of the International Union of Biochemistry and Molecular Biology (IUBMB)) include proteases within this group.

Examples include proteases selected from those classified under the Enzyme Classification (E.C.) numbers:

3.4.11 (i.e. so-called aminopeptidases), including 3.4.11.5 (Prolyl aminopeptidase), 3.4.11.9 (X-pro aminopeptidase), 3.4.11.10 (Bacterial leucyl aminopeptidase), 3.4.11.12 (Thermophilic aminopeptidase), 3.4.11.15 (Lysyl aminopeptidase), 3.4.11.17 (Tryptophanyl aminopeptidase), 3.4.11.18 (Methionyl aminopeptidase).

3.4.21 (i.e. so-called serine endopeptidases), including 3.4.21.1 (Chymotrypsin), 3.4.21.4 (Trypsin), 3.4.21.25 (Cucumisin), 3.4.21.32 (Brachyurin), 3.4.21.48 (Cerevisin) and 3.4.21.62 (Subtilisin);

3.4.22 (i.e. so-called cysteine endopeptidases), including 3.4.22.2 (Papain), 3.4.22.3 (Ficain), 3.4.22.6 (Chymopapain), 3.4.22.7 (Asdepain), 3.4.22.14 (Actinidain), 3.4.22.30 (Caricain) and 3.4.22.31 (Ananain);

3.4.23 (i.e. so-called aspartic endopeptidases), including 3.4.23.1 (Pepsin A), 3.4.23.18 (Aspergillopepsin I), 3.4.23.20 (Penicillopepsin) and 3.4.23.25 (Saccharopepsin); and

3.4.24 (i.e. so-called metalloendopeptidases), including 3.4.24.28 (Bacillolysin).

Examples of relevant subtilisins comprise subtilsin BPN′, subtillsin amylosacchariticus, subtilisin 168, subtilisin mesentericopeptidase, subtilisin Carlsberg, subtlisin DY, subtilisin 309, subtilisin 147, thermitase, aqualysin, Bacillus PB92 protease, proteinase K, Protease TW7, and Protease TW3.

Specific examples of such readily available commercial proteases include Esperase®, Alcalase®, Neutrase®, Dyrazym®, Savinase®, Pyrase®, Pancreatic Trypsin NOVO (PTN), Bio-Feed® Pro, Clear-Lens Pro® (all enzymes available from Novozymes A/S).

Examples of other commercial proteases include Maxtase®, Maxacal®, Maxapem® marketed by Gist-Brocades N.V., Opticlean® marketed by Solvay et Cle. and Purafect® marketed by Genencor International.

It is to be understood that also protease variants are contemplated as the parent protease. Examples of such protease variants are disclosed in EP 130.756 (Genentech), EP 214.435 (Henkel), WO 87/04461 (Amgen), WO 87/05050 (Genex), EP 251.446 (Genencor), EP 260.105 (Genencor), Thomas et al., (1985), Nature. 318, p. 375-376, Thomas et al., (1987), J. Mol. Biol., 193, pp. 803-813, Russel et al., (1987), Nature, 328, p. 496-500, WO 88/08028 (Genex), WO 88/08033 (Amgen), WO 89/06279 (Novo Nordisk A/S), WO 91/00345 (Novo Nordisk A/S), EP 525 610 (Solvay) and WO 94/02618 (Gist-Brocades N.V.). The activity of proteases can be determined as described in “Methods of Enzymatic Analysis”, third edition, 1984, Verlag Chemie, Weinheim, vol. 5.

Parent Lipases

Parent lipases (i.e. enzymes classified under the Enzyme Classification number E.C. 3.1.1 (Carboxylic Ester Hydrolases) in accordance with the Recommendations (1992) of the International Union of Biochemistry and Molecular Biology (IUBMB)) include lipases within this group.

Examples include lipases selected from those classified under the Enzyme Classification (E.C.) numbers:

3.1.1 (i.e. so-called Carboxylic Ester Hydrolases), including (3.1.1.3) Triacylglycerol I lipases, (3.1.1.4.) Phosphorlipase A2.

Examples of lipases include lipases derived from the following microorganisms: Humicola, e.g. H. brevispora, H. lanuginosa, H. brevis var. thermoldea and H. insolens (U.S. Pat. No. 4,810,414).

Pseudomonas, e.g. Ps. fragi, Ps. stutzeri, Ps. cepada and Ps. fluorescens (WO 89/04361), or Ps. plantarii or Ps. gladioli (U.S. Pat. No. 4,950,417 (Solvay enzymes)) or Ps. alcaligenes and Ps. pseudoalcaligenes (EP 218 272) or Ps. mendodna (W/O 88/09367; U.S. Pat. No. 5,389,536).

Fusarium, e.g. F. oxysporum (EP 130,064) or F. solani pisl (WO 90/09446).

Mucor (also called Rhizomucor), e.g. M. miehei (EP 238 023).

Chromobacterium (especially C. viscosum). Aspergillus (especially A. niger).

Candida, e.g. C. cylindracea (also called C. rugosa) or C. antarctica (WO 88/02775) or C. antarctica lipase A or B (WO 94/01541 and WO 89/02916).

Geotricum, e.g. G. candidum (Schimada et al., (1989), J. Biochem., 106, 383-388).

Penicillium, e.g. P. camembertil (Yamaguchi et al., (1991), Gene 103, 61-67).

Rhizopus, e.g. R. delemar (Hass et al., (1991), Gene 109, 107-113) or R. niveus (Kugimlya et al., (1992) Biosci. Biotech. Biochem 56, 716-719) or R. oryzae.

Bacillus, e.g. B. subtilis (Dartois et al., (1993) Biochemica et Biophysica acta 1131, 253-260) or B. stearothermophilus (JP 64/7744992) or B. pumilus (WO 91/16422).

Specific examples of readily available commercial lipases include Lipolase®, Lipolase® Ultra, Lipozyme®, Palatase), Novozym® 435, Lecitase® (all available from Novozymes A/S). Examples of other lipases are Lumafast®, Ps. mendocian lipase from Genencor Int. Inc.; Lipomax®, Ps. pseudoalcaligenes lipase from Gist Brocades/Genencor Int. Inc.; Fusarium solani lipase (cutinase) from Unilever; Bacillus sp. lipase from Solvay enzymes. Other lipases are available from other companies.

It is to be understood that also lipase variants are contemplated as the parent enzyme. Examples of such are described in e.g. WO 93/01285 and WO 95/22615.

The activity of the lipase can be determined as described in “Methods of Enzymatic Analysis”, Third Edition, 1984, Veriag Chemie, Weinhein, vol. 4, or as described in AF 95/5 GB (available on request from Novozymes A/S).

Parent Oxidoreductases

Parent oxidoreductases (i.e. enzymes classified under the Enzyme Classification number E.C. 1 (Oxidoreductases) in accordance with the Recommendations (1992) of the International Union of Biochemistry and Molecular Biology (IUBMB)) include oxidoreductases within this group.

Examples include oxidoreductases selected from those classified under the Enzyme Classification (E.C.) numbers:

Glycerol-3-phosphate dehydrogenase _NAD+_(1.1.1.8), Glycerol-3-phosphate dehydrogenase _NAD(P)+_(1.1.1.94), Glycerol-3-phosphate 1-dehydrogenase NADP_(1.1.1.94), Glucose oxidase (1.1.3.4), Hexose oxidase (1.1.3.5), Catechol oxidase (1.1.3.14), Billrubin oxidase (1.3.3.5), Alanine dehydrogenase (1.4.1.1), Glutamate dehydrogenase (1.4.1.2), Glutamate dehydrogenase _NAD(P)+_(1.4.1.3), Glutamate dehydrogenase _NADP+_(1.4.1.4), L-Amino acid dehydrogenase (1.4.1.5), Serine dehydrogenase (1.4.1.7), Valine dehydrogenase _NADP+_(1.4.1.8), Leucine dehydrogenase (1.4.1.9), Glycine dehydrogenase (1.4.1.10), L-Amino-acid oxidase (1.4.3.2.), D-Amino-acid oxidase(1.4.3.3), L-Glutamate oxidase (1.4.3.11), Protein-lysine 6-oxidase (1.4.3.13), L-lysine oxidase (1.4.3.14), L-Aspartate oxidase (1.4.3.16), D-amino-acid dehydrogenase (1.4.99.1), Protein disulfide reductase (1.6.4.4), Thioredoxin reductase (1.6.4.5), Protein disulfide reductase (glutathione) (1.8.4.2), Laccase (1.10.3.2), Catalase (1.11.1.6), Peroxidase (1.11.1.7), Upoxygenase (1.13.11.12), Superoxide dismutase (1.15.1.1)

Said Glucose oxidases may be derived from Aspergillus niger. Said Laccases may be derived from Polyporus pinsitus, Myceliophtora thermophila, Coprinus cinereus, Rhizoctonia solani, Rhizoctonia praticola, Scytalidium thermophilum and Rhus vernicifera. Bilirubin oxidases may be derived from Myrothechecium verrucaria. The Peroxidase may be derived from e.g. Soy bean, Horseradish or Coprinus cinereus. The Protein Disulfide reductases Protein Disulfide reductases of bovine origin, Protein Disulfide reductases derived from Aspergillus oryzae or Aspergillus niger, and DsbA or DsbC derived from Escherichia coli.

Specific examples of readily available commercial oxidoreductases include Gluzyme (enzyme available from Novozymes A/S). However, other oxidoreductases are available from others. It is to be understood that also variants of oxidoreductases are contemplated as the parent enzyme.

The activity of oxidoreductases can be determined as described in “Methods of Enzymatic Analysis”, third edition, 1984, Verlag Chemie, Weinheim, vol. 3.

Parent Carbohydrases

Parent carbohydrases may be defined as all enzymes capable of breaking down carbohydrate chains (e.g. starches) of especially five and six member ring structures (i.e. enzymes classified under the Enzyme Classification number E.C. 3.2 (glycosidases) in accordance with the Recommendations (1992) of the International Union of Biochemistry and Molecular Biology (IUBMB)).

Examples include carbohydrases selected from those classified under the Enzyme Classification (E.C.) numbers:

alfa-amylase (3.2.1.1) alfa-amylase (3.2.1.2), glucan 1,4-alfa-glucosidase (3.2.1.3), cellulase (3.2.1.4), endo-1,3(4)-beta-glucanase (3.2.1.6), endo-1,4-beta-xylanase (3.2.1.8), dextranase (3.2.1.11), chitinase (3.2.1.14), polygalacturonase (3.2.1.15), lysozyme (3.2.1.17), beta-glucosidase (3.2.1.21), alfa-galactosidase (3.2.1.22), beta-galactosidase (3.2.1.23), amylo-1,6-glucosidase (3.2.1.33), xylan 1,4-beta-xylosidase (3.2.1.37), glucan endo-1,3-beta-D-glucosidase (3.2.1.39), alfa-dextrin endo-1,6-glucosidase (3.2.1.41), sucrose alfa-glucosidase (3.2.1.48), glucan endo-1,3-alfa-glucosidase (3.2.1.59), glucan 1,4-beta-glucosidase (3.2.1.74), glucan endo-1,6-beta-glucosidase (3.2.1.75), arabinan endo-1,5-alfa-arabinosidase (3.2.1.99), lactase (3.2.1.108), and chitonanase (3.2.1.132).

Specific examples of readily available commercial carbohydrases include Alpha-Gal®, Bio-Feed® Alpha, Bio-Feed® Beta, Bio-Feed® Plus, Bio-Feed® Plus, Novozyme® 188, Carezyme®, Celluclast®, Cellusoft®, Ceremyl®, Citrozym®, Denimax®, Dezyme®, Dextrozyme®, Finizym®, Fungamyl®, Gamanase®, Glucanex®, Lactozym®, Maltogenase®, Pentopan®, Pectinex®, Promozyme®, Pulpzyme®, Novamyl®, Termamyl®, AMG (Amyloglucosidase Novo), Maltogenase®, Aquazym®, Natalase® (all enzymes available from Novozymes A/S). Other carbohydrases are available from other companies.

It is to be understood that also carbohydrase variants are contemplated as the parent enzyme. The activity of carbohydrases can be determined as described in “Methods of Enzymatic Analysis”, third edition, 1984, Verlag Chemie, Weinheim, vol. 4.

Parent Transferases

Parent transferases (i.e. enzymes classified under the Enzyme Classification number E.C. 2 in accordance with the Recommendations (1992) of the International Union of Biochemistry and Molecular Biology (IUBMB)) include transferases within this group. The parent transferases may be any transferase in the subgroups of transferases: transferases transferring one-carbon groups (E.C. 2.1); transferases transferring aldehyde or residues (E.C 2.2); acyltransferases (E.C. 2.3); glucosyltransferases (E.C. 2.4); transferases transferring alkyl or aryl groups, other that methyl groups (E.C. 2.5); transferases transferring nitrogeneous groups (2.6).

In a preferred embodiment the parent transferase is a transglutaminase E.C 2.3.2.13(Protein-glutamine beta-glutamyltransferase).

Transglutaminases are enzymes capable of catalyzing an acyl transfer reaction in which a gamma-carboxyamide group of a peptide-bound glutamine residue is the acyl donor. Primary amino groups in a variety of compounds may function as acyl acceptors with the subsequent formation of monosubstituted gamma-amides of peptide-bound glutamic acid. When the epsilon-amino group of a lysine residue in a peptide-chain serves as the acyl acceptor, the transferases form intramolecular or intermolecular gamma-glutamyl-epsilon-lysyl crosslinks. The parent transglutaminase may be of human, animal (e.g. bovine) or microbial origin. Examples of such parent transglutaminases are animal derived Transglutaminase, FXIIIa; microbial transglutaminases derived from Physarum polycephalum (Klein et al., Journal of Bacteriology, Vol. 174, p. 2599-2605); transglutaminases derived from Streptomyces sp., including Streptomyces lavendulae, Streptomyces lydicus (former Streptomyces libani) and Streptoverticillium sp., including Streptoverticillium mobaraense, Streptoverticillium cinnamoneum, and Streptoverticillium griseocameum (Motoki et al., U.S. Pat. No. 5,156,956; Andou et al., U.S. Pat. No. 5,252,469; Kaempfer et al., Journal of General Microbiology, Vol. 137, p. 1831-1892; Ochi et al., International Journal of Systematic Bacteriology, Vol. 44, p. 285-292; Andou et al., U.S. Pat. No. 5,252,469; Williams et al., Journal of General Microbiology, Vol. 129, p. 1743-1813).

It is to be understood that also transferase variants are contemplated as the parent enzyme. The activity of transglutaminases can be determined as described in “Methods of Enzymatic Analysis”, third edition, 1984, Veriag Chemie, Weinheim, vol. 1-10.

Parent Phytases

Parent phytases are included in the group of enzymes classified under the Enzyme Classification number E.C. 3.1.3 (Phosphoric Monoester Hydrolases) in accordance with the Recommendations (1992) of the International Union of Biochemistry and Molecular Biology (IUBMB)).

Phytases are enzymes produced by microorganisms, which catalyse the conversion of phytate to inositol and inorganic phosphorus.

Phytase producing microorganisms comprise bacteria such as Bacillus subtilis, Bacillus natto and Pseudomonas; yeasts such as Saccharomyces cerevisiae; and fungi such as Aspergillus niger, Aspergillus ficuum, Aspergillus awamori, Aspergillus oryzae, Aspergillus terreus or Aspergillus nidulans, and various other Aspergillus species).

Examples of parent phytases include phytases selected from those classified under the Enzyme Classification (E.C.) numbers: 3-phytase (3.1.3.8) and 6-phytase (3.1.3.26).

The activity of phytases can be determined as described in “Methods of Enzymatic Analysis”, third edition, 1984, Veriag Chemie, Weinheim, vol. 1-10, or may be measured according to the method described in EP-A1-0 420 358, Example 2A.

Lyases

Suitable lyases include Polysaccharide lyases: Pectate lyases (4.2.2.2) and pectin lyases (4.2.2.10), such as those from Bacillus licheniformis disclosed in WO 99/27083.

Isomerases

Protein Disulfide Isomerase.

Without being limited thereto suitable protein disulfide isomerases include PDIs described in WO 95/01425 (Novo Nordisk A/S) and suitable glucose isomerases include those described in Biotechnology Letter, Vol. 20, No 6, June 1998, pp. 553-56.

Contemplated isomerases include xylose/glucose isomerase (5.3.1.5) including Sweetzyme® (available from Novozymes A/S).

Materials and Methods

Strains and Plasmids

E. coli DH12S (available from Gibco BRL) is used for yeast plasmid rescue.

pTMPP2ver2 is a S. cerevisiae and E. coli shuttle vector under the control of TPI promoter, constructed from pJC039 described in WO 00/10038. It is used for library construction, yeast expression, screening and sequencing.

Saccharomyces cerevsiae YNG318: MATa Dpep4[cir+] ura3-52, leu2-D2, his 4-539 is used for the construction of yeast library and the expression of the fusion protein. It is described in J. Biol. Chem. 272 (15), 9720-9727, 1997).

Media and Substrates

10× Basal Solution

| | | |
|---|---|---|
| 66.8 | g/L | Yeast nitrogen base with out |
| | | amino acids (DIFCO) |
| 100 | g/L | succinate |
| 60 | g/L | NaOH |

SC-Glucose

| | | |
|---|---|---|
| 100 | mL/L | 20% glucose (i.e., a final concentration |
| | | of 2% = 2 g/100 ml)) |
| 4 | mL/L | 5% threonine |
| 10 | mL/L | 1% tryptophan |
| 25 | mL/L | 20% casamino acids |
| 100 | mL/L | 10 X basal solution |

The above solution is sterilized using a filter of a pore size of 0.20 micro meters. Agar and H2O (approx. 761 ml) is autoclaved together, and the separately sterilized SC-glucose solution is added to the agar solution.

YPD

| | | |
|---|---|---|
| 20 | g/L | Bacto pepton |
| 10 | g/L | yeast extract |
| 100 | mL/L | 20% glucose |
| | | (sterilized separately) |

N-Phytate Plate

| | | |
|---|---|---|
| 100 | mL/L | 1M Na acetate buffer (pH 5.5) |
| 5 | g/L | Na phytate |
| 30 | g/L | agar |

PEG/LIAc Solution

| | |
|---|---|
| 50 mL | 40% PEG4000 |
| | (sterilized by autoclaving) |
| 1 mL | 5M Lithium Acetate |
| | (sterilized by autoclaving) |

Trace Metal Solution

| | | |
|---|---|---|
| FeSO4 × 7H2O | 13.90 | g/L |
| MnSO4 × 5 H2O | 13.60 | g/L |
| ZnCl2 | 6.80 | g/L |
| CuSO4 × 5 H2O | 2.50 | g/L |
| NiCl2 × 6 H2O | 0.24 | g/L |
| Citric acid × H2O | 3.00 | g/L |

pNPB

p-nitrophenyl butyrate (SIGMA N-9876)

Cutinase Activity (LU)

A substrate for cutinase is prepared by emulsifying tributyrin (glycerin tributyrate) using gum Arabic as emulsifier. The hydrolysis of tributyrin at 30° C. at pH7 is followed in a pH-stat titration experiment. One unit of cutinase activity (1LU) equals the amount of enzyme capable of releasing 1 micro mol butyric acid/min at the standard conditions.

Phytase Activity Assay

10 micro L diluted enzyme samples (diluted in 0.1 M sodium acetate, 0.01% Tween20, pH 5.5) were added into 250 micro L 5 mM sodium phytate (Sigma) in 0.1 M sodium acetate, 0.01% Tween20, pH 5.5 (pH adjusted after dissolving the sodium phytate; the substrate was preheated) and incubated for 30 minutes at 37° C. The reaction was stopped by adding 250 micro L 10% TCA and free phosphate was measured by adding 500 micro L 7.3 g FeSO4 in 100 ml molybdate reagent (2.5 g (NH4)6Mo7O24.4H2O in 8 ml H2SO4 diluted to 250 ml). The absorbance at 750 nm was measured on 200 micro L samples in 96 well microtiter plates. Substrate and enzyme blanks were included. A phosphate standard curve was also included (0-2 mM phosphate). I FYT equals the amount of enzyme that releases 1 micromole phosphate/min at the given conditions.

### EXAMPLES

### Example 1

### Construction of Nucleic Add Sequence Encoding Fusion Protein

The cutinase gene was amplified by PCR using the below primers AM34 (SEQ ID NO: 1) and Cutd-R (SEQ ID NO: 2). The phytase gene together with the linker region was amplified by PCR using the primers Cuti-linker-P (SEQ ID NO: 3) and AM35 (SEQ ID NO: 4).

PCR is carried out by the PTC-200 DNA Engine. DNA fragments are recovered from agarose gel by the Qiagen gel extraction Kit. The resulted two fragments were Joined by SOE method (Splicing by Overlap Extension, see “PCR: A practical approach”, p. 207-209, Oxford University press, eds. McPherson, Quirke, Taylor). The PCR conditions are as follows:

| PCR reaction system: | | | Conditions: | | |
|---|---|---|---|---|---|
| 38.9 | micro L | H2O | 1 | 98° C. | 10 sec |
| 5 | micro L | 10 X reaction buffer | 2 | 68° C. | 90 sec |
| 1 | micro L | Klen Taq LA | 1-2 | 30 cycles | |
| | | (CLONTECH) | | | |
| 4 | micro L | 10 mM dNTPs | 3 | 68° C. | 10 min |
| 0.3 | micro L X 2 | 100 pmole/micro | | | |
| | | L Primers | | | |
| 0.5 | micro L | Template DNA | | | |

The resulting fragments were gel-purified and used for the template for the second PCR reaction.

| PCR reaction system: | | | Conditions: | |
|---|---|---|---|---|
| 38.4 | micro L | H2O | 1 | 98° C. |
| 5 | micro L | 10 X reaction buffer | | 10 sec |
| 1 | micro L | Klen Taq LA (CLONTECH) | 2 | 66° C. |
| 4 | micro L | 10 mM dNTPs | | 90 sec |
| 0.3 | micro L X 2 | 100 pmole/micro L Primers | 1-2 | 30 cycles |
| 0.5 | micro L X 2 | PCR fragments | 3 | 66° C. |
| | | | | 10 min |

### Example 2

### Transformation and Expression of the Fusion Protein in S. cerevisiae

The S. cerevisiae transformants were obtained by the following procedure:
1. Mix 0.5 micro L of vector (Xba I digested) and 1 micro L of PCR fragments obtained in Example 1.
2. Thaw YNG318 competent cells on ice and use as host cell.
3. Mix 100 micro L of the cells, the DNA mixture from step 1 above and 10 micro L of carrier DNA (Clontech) in 12 ml polypropylene tubes (Falcon 2059).
4. Add 0.6 ml PEG/LIAc solution and mix gently.
5. Incubate for 30 min at 30° C., and 200 rpm.
6. Incubate for 30 min at 42° C. (heat shock).
7. Transfer to an eppendorf tube and centrifuge for 5 sec.
8. Remove the supernatant and resolve sediment in 3 ml of YPD.
9. Incubate the cell suspension for 45 min at 200 rpm at 30° C.
10. Pour the suspension to SC-lucose plates and incubate at 30° C. for 3 days.

The obtained transformants were cultivated in YPD medium in 24 well plates at 25° C. for 3 days at 180 rpm.

The plates were centrifuged and the supernatant was assayed for cutinase activity and phytase activity.

| Transformant | Cutinase activity | Phytase activity | Ratio |
|---|---|---|---|
| No | (LU/ml) | (FYT/ml) | (FYT/LU) |
| 1 | 4.36 | 7.00 | 1.6 |
| 2 | 2.59 | 4.65 | 1.8 |
| 3 | 4.29 | 6.25 | 1.5 |
| 4 | 0.68 | 1.65 | 2.4 |
| 5 | 2.87 | 5.00 | 1.7 |

The transformants showed both cutinase and phytase activity meaning that the fusion protein was secreted and folded properly as an active form. Further the table shows that the activity ratio is at a constant level and that the two enzymes consequently must be co-expressed as a fused enzyme.

### Example 3

### Comparison of Relative Specific Activity Using Phytase Variants

Two kinds of fusion protein using two phytase variants genes in combination with one cutinase were constructed by the method as described in example 1. The two phytase variants are denoted Variant N and Variant X.

The specific activity ratio of Variant N to Variant X is 100:180. The cutinase and phytase activities of several transformants were measured.

| Cutinase | Phytase | Cutinase activity | Phytase activity | Ratio |
|---|---|---|---|---|
| variant | variant | (LU/ml) | (FYT/ml) | (FYT/LU) |
| Variant A | Variant N | 1.65 | 0.68 | 0.41 |
| Variant A | Variant N | 4.65 | 2.59 | 0.56 |
| Variant A | Variant N | 6.25 | 4.29 | 0.69 |
| Variant A | Variant N | 7.00 | 4.36 | 0.62 |
| Variant A | Variant N | 5.00 | 2.87 | 0.57 |
| Variant A | Variant N | 8.00 | 5.32 | 0.67 |
| Variant A | Varlant X | 10.00 | 11.15 | 1.12 |
| Variant A | Variant X | 7.30 | 9.31 | 1.28 |
| Variant A | Variant X | 13.00 | 17.64 | 1.36 |
| Variant A | Variant X | 9.00 | 10.91 | 1.21 |
| Variant A | Variant X | 3.75 | 3.70 | 0.99 |
| Variant A | Variant X | 2.50 | 2.93 | 1.17 |
| Variant A | Varlant X | 8.75 | 11.88 | 1.36 |

Data shows that the activity-ratio is at a constant level for each of the two variant combinations and that the two enzymes consequently must be co-expressed as a fused enzyme. Further the ratio between FYT/LU for the two fused enzymes systems is close to the expected value of 1.8, and it can consequently be concluded that a change in specific activity can be monitored by measuring the activity ratio of the fused enzyme.

### Example 4

### Other Linker (FLAG)

The fusion protein with another linker FLAG (DYKDDDK) was constructed using the same method as described in the example 1. The following primers were used for SOE: AM34 (SEQ ID NO: 1), Cuti-R (SEQ ID NO: 2), AM35 (SEQ ID NO:4), and Cuti-FLAG-P (SEQ ID NO:5).

The transformant was cultured and assayed for cutinase and phytase activities as described in example 2.

| | Cutinase activity | Phytase activity | Ratio |
|---|---|---|---|
| Linker | (LU/ml) | (FYT/ml) | (FYT/LU) |
| FLAG | 3.63 | 5.18 | 1.43 |
| PEPTPEPT | 4.31 | 6.22 | 1.44 |

The ratio between phytase activity and cutinase activity is constant, and at the same level as in example 2. It can consequently be concluded that the two enzymes are co-expressed as a fused enzyme independent of choice of linker.

### Example 5

### High Through Put Screening

Relative activities of cutinase and phytase activities were measured in the same well of 96-well micro tier plates using the following method. In this example the two proteins as described in the example 3 were used (variant A+variant X, variant A+variant N).

Method:

1. Add 2.5 micro L of samples at several concentrations of the fusion protein in a 96-well micro plate.

2. Add 100 micro L of substrate solution.

Substrate solution:
A. 1 ml of 3 mg/ml pNPB dissolved in 2-propanol.
B. 10 ml of 1 mg/ml Na-phytate solution dissolved in 0.1M acetate buffer (pH 5.75) Mix A and B just before experiment.
   3. Incubate at room temperature for 10 minutes.
   4. Measure A405 (Cutinase activity)
   5. Add 100 micro L of stop solution (7.3 g FeSO4 in 100 ml molybdate reagent (2.5 g (NH4)6Mo7O24.4H2O in 8 ml H2SO4 diluted to 250 ml) and keep the plate still for 10 minutes.

6. Measure A750 (Phytase activity) and calculate the ratio (A750/A405).

| Well | Cutinase | Phytase | | | Ratio |
|---|---|---|---|---|---|
| No. | variant | variant | A405 | A750 | (A750/A405) |
| 1 | Variant A | Variant N | 0.155 | 0.108 | 0.70 |
| 2 | Variant A | Variant N | 0.243 | 0.162 | 0.67 |
| 3 | Variant A | Variant N | 0.295 | 0.201 | 0.68 |
| 4 | Variant A | Variant N | 0.385 | 0.299 | 0.78 |
| 5 | Variant A | Variant X | 0.175 | 0.193 | 1.10 |
| 6 | Variant A | Variant X | 0.251 | 0.252 | 1.00 |
| 7 | Variant A | Variant X | 0.319 | 0.298 | 0.93 |
| 8 | Variant A | Variant X | 0.387 | 0.397 | 1.03 |

The table shows that the relative each enzyme activity is possible to be measured in one well of 96-well micro titre plates and that variants with improved specific activity can be screened.

1-16. (canceled)

17. A method of screening enzymes for variants with improved specific activity, comprising the steps of
(i) generating a library of nucleic acid sequences encoding enzyme variants of interest
(ii) providing a nucleic acid sequence encoding an enzyme to be fused with the enzyme in (i)
(iii) fusing nucleic acid sequence encoding enzyme variants in (i) with nucleic acid sequence encoding enzyme in (ii)
(iv) transforming the fused nucleic acid sequence obtained in (iii) into a host cell
(v) culturing host cell in (iv) in order to express the fused enzymes
(vi) sampling each cell culture obtained in (v)
(vii) analyzing samples obtained in (vi) by determining activity ratio of the expressed fused enzymes
(viii) selecting the samples exhibiting the desired activity ratio.

18. The method of claim 17, wherein the enzymes are fused by means of a linker by fusing nucleic acid sequence encoding enzyme variants in 1(i) with nucleic acid sequence encoding a linker and further with nucleic acid sequence encoding enzyme in 1(ii).

19. The method of claim 18, wherein the linker consists of 1-40, or 2-20, or 2-10 amino acids.

20. The method of claim 18, wherein the linker is selected from the group consisting of Poly-Arg, Poly-His, PEPTPEPT, FLAG, Strep-tag II, c-myc, S-, HAT-, 3× FLAG, Calmoludin-binding peptide, Cellulose-binding domain, SBP, Chitin-binding domain, Glutathione S-transferase, Maltose-binding domain.

21. The method of claim 17, wherein the library is generated by mutating a nucleic acid sequence encoding a wild type enzyme.

22. The method of claim 17, wherein the library is generated by mutating a nucleic acid sequence encoding a protein engineered enzyme.

23. The method of claim 17, wherein the enzyme variant in 1(i) is generated by genetic engineering.

24. The method of claim 21, wherein the enzyme is selected from the group consisting of proteases, cellulases (endoglucanases), β-glucanases, hemicellulases, lipases, peroxidases, laccases, α-amylases, glucoamylases, cutinases, pectinases, reductases, oxidases, phenoloxidases, ligninases, pullulanases, pectate lyases, xyloglucanases, xylanases, pectin acetyl esterases, polygalacturonases, rhamnogalacturonases, pectin lyases, mannanases, pectin methylesterases, cello-biohydrolases, transglutaminases and phytases.

25. The method of claim 17, wherein the enzyme in 1(ii) is selected from the group consisting of proteases, cellulases (endoglucanases), β-glucanases, hemicellulases, lipases, peroxidases, laccases, α-amylases, glucoamylases, cutinases, pectinases, reductases, oxidases, phenoloxidases, ligninases, pullulanases, pectate lyases, xyloglucanases, xylanases, pectin acetyl esterases, polygalacturonases, rhamnogalacturonases, pectin lyases, mannanases, pectin methylesterases, cello-biohydrolases, transglutaminases and phytases.

26. The method of claim 17, wherein the host cells in 1(iv) are selected from bacterial cells.

27. The method of claim 26, wherein the host cells belong to a strain selected from the group consisting of the species Bacillus alkalophilus, Bacillus agaradhaerens, Bacillus amyloliquefaciens, Bacillus brevis, Bacillus clausii, Bacillus circulans, Bacillus coagulans, Bacillus lautus, Bacillus lentus, Bacillus licheniformis, Bacillus megaterium, Bacillus stearothermophilus, Bacillus subtilis, Bacillus thuringiensis, Streptomyces lividans and Streptomyces murinus.

28. The method of claim 17, wherein the host cells in 1(iv) are selected from fungal cells.

29. The method of claim 28, wherein the host cells belong to a strain selected from the group consisting of the genera Acremonium, Aspergillus, Fusarium, Humicola, Myceliophthora, Neurospora, Penicillium, Thielavia, Tolypocladium, Trichoderma, Eupenicillium, Emericella, Eurotium, Allomyces, Blastocladiella, Coelomomyces, Achlya, Candida, Alternaria, Rhizopus and Mucor; preferably the species Aspergillus awamori, Aspergillus foetidus, Aspergillus japonicus, Aspergillus niger, Aspergillus nidulans or Aspergillus oryzae.

30. The method of claim 17, wherein the host cells in 1(iv) are selected from yeast cells.

31. The method of claim 30, wherein the host cells belong to a strain selected from the group consisting of the genera Candida, Kluyveromyces, Saccharomyces, Schizosaccharomyces, Candida, Pichia, Hansehula, or Yarrowia, preferably to the species Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, Kluyveromyces lactis, Kluyveromyces fragilis, Hansenula polymorpha, Pichia pastoris Yarrowia lipolytica, Schizosaccharomyces pombe, Ustilgo maylis, Candida maltose, Pichia guillermondii and Pichia methanolio.

32. The method of claim 1, wherein the fused enzymes in 1(v) are an extracellular product.
